(19) United States
(12) Patent Application Publication
(10) Pub. No.: US 2026/0315379 A1
(43) Pub. Date: Oct. 15, 2026

(54) KIT AND METHOD FOR NASAL STEROID IRRIGATION

(71) Applicant: 4B Healthcare Inc., Murfreesboro, TN (US)

(72) Inventors: Brannon Mangus, Murfreesboro, TN (US); Matthew Stearns, Murfreesboro, TN (US)

(21) Appl. No.: 19/179,432

(22) Filed: Apr. 15, 2025

(51) Int. Cl. A61K 9/00; A61K 31/55; A61K 31/58; A61K 33/10; A61K 33/14

(52) U.S. Cl. CPC A61K 9/009; A61K 9/0043; A61K 31/55; A61K 31/58; A61K 33/10; A61K 33/14

(57) ABSTRACT

The present disclosure relates to a method for providing a nasal treatment, comprising providing a partitioned sachet with a plurality of active ingredients, the plurality of active ingredients contained within at least two anhydrous dosing compartments; with the various active ingredients including sodium bicarbonate, sodium chloride, a steroid, and/or an antihistamine.

### FIELD OF THE DISCLOSURE

The present disclosure relates generally to nasal irrigation, and more particularly to a sachet used for maintaining doses for use in nasal irrigation.

### BACKGROUND

Intranasal administration of pharmaceutical compositions has been explored through various approaches, primarily focusing on the delivery mechanisms and formulation stability. Known methods have often utilized liquid formulations delivered via nasal sprays or droppers, which can be prone to issues such as leakage, imprecise dosing, and degradation of active ingredients due to exposure to moisture. These liquid-based systems typically require preservatives to maintain stability, which can lead to irritation of the nasal mucosa and limit the types of active ingredients that can be effectively delivered.

Another approach has involved the use of powder-based formulations for intranasal delivery. These systems aim to overcome the limitations of liquid formulations by providing a more stable environment for active ingredients. However, challenges remain in ensuring uniform distribution of the powder within the nasal cavity and achieving consistent dosing. Devices designed for powder delivery often require complex mechanisms to aerosolize the powder, which can increase the cost and complexity of the device. Additionally, the hygroscopic nature of many pharmaceutical powders necessitates careful packaging and handling to prevent moisture absorption, which can compromise the efficacy of the active ingredients.

These approaches fail to provide an easy to use and stable system for dual nasal irrigation and steroid administration. Various kits and methods related to the administration of a nasal irrigation and steroid treatment to a subject in need thereof are provided according to various aspects of the present disclosure.

### BRIEF SUMMARY

The current disclosure provides an enhancement to conventional nasal irrigation and steroid treatments, at least in part by introducing a novel kit for intranasal administration of pharmaceutical compositions and methods utilizing the same.

In some aspects, the techniques described herein relate to a method for providing a nasal treatment, including the steps of: providing a partitioned sachet with a plurality of active ingredients, the plurality of active ingredients contained within at least two anhydrous dosing compartments; and administering the plurality of active ingredients to an individual.

In some aspects, the techniques described herein relate to a method, wherein: the step of providing a partitioned sachet further includes a partitioned sachet with a first anhydrous dosing compartment with a sodium bicarbonate dosage and a second anhydrous dosing compartment with a steroid dosage and sodium chloride dosage, the first anhydrous dosing compartment and the second anhydrous dosing compartment separated by a middle barrier layer; and the step of administering the plurality of active ingredients further includes administering the sodium bicarbonate dosage, steroid dosage, and sodium chloride dosage.

In some aspects, the techniques described herein relate to a method, further including: the step of administering the sodium bicarbonate dosage, steroid dosage, and sodium chloride dosage includes administering the dosages together.

In some aspects, the techniques described herein relate to a method, further including: the step of providing a partitioned sachet further includes providing a partitioned sachet with a tear feature; and the step of administering the sodium bicarbonate dosage, steroid dosage, and sodium chloride dosage together further includes engaging the tear feature to create an opening of at least one of the first anhydrous dosing compartment and the second anhydrous dosing compartment.

In some aspects, the techniques described herein relate to a method wherein the step of administering the sodium bicarbonate dosage, steroid dosage, and sodium chloride dosage together includes: simultaneously pouring out the sodium bicarbonate dosage of the first anhydrous dosing compartment and the steroid dosage, and sodium chloride dosage of the second anhydrous dosing compartment through the opening in the first anhydrous dosing compartment and the second anhydrous dosing compartment created by engaging the tear feature.

In some aspects, the techniques described herein relate to a method, wherein: the sodium bicarbonate dosage is between about 300 milligrams and 1000 milligrams; and the sodium chloride dosage is between about 1400 milligrams and 2500 milligrams.

In some aspects, the techniques described herein relate to a method wherein the sodium chloride dosage relative to the sodium bicarbonate dosage is in a ratio of about 3:1.

In some aspects, the techniques described herein relate to a method, wherein the steroid dosage is chosen from budesonide, mometasone, triamcinolone, fluticasone, and combinations thereof.

In some aspects, the techniques described herein relate to a method, wherein the steroid dosage is between about 200 micrograms and about 5000 micrograms.

In some aspects, the techniques described herein relate to a method further including an antihistamine in a range between about 250 micrograms to about 50000 micrograms.

In some aspects, the techniques described herein relate to a method wherein the antihistamine is chosen from azelastine, olopatadine and combinations thereof.

In some aspects, the techniques described herein relate to a method, wherein: the step of providing a partitioned sachet further includes a partitioned sachet with a first anhydrous dosing compartment with a sodium bicarbonate dosage and a second anhydrous dosing compartment with an antihistamine dosage and a sodium chloride dosage, the first anhydrous dosing compartment and the second anhydrous dosing compartment separated by a middle barrier layer; and the step of administering the plurality of active ingredients further includes administering the sodium bicarbonate dosage, the antihistamine dosage, and the sodium chloride dosage.

In some aspects, the techniques described herein relate to a kit for intranasal administration of a composition, the kit including: at least one sachet including one or more anhydrous dosing compartments; wherein the composition includes a plurality of anhydrous active ingredients.

In some aspects, the techniques described herein relate to a kit, wherein the one or more anhydrous dosing compartments includes a first anhydrous dosing compartment and a second anhydrous dosing compartment separated by a middle barrier layer.

In some aspects, the techniques described herein relate to a kit, wherein: the plurality of anhydrous active ingredients includes a sodium bicarbonate, a steroid, and a sodium chloride.

In some aspects, the techniques described herein relate to a kit, wherein: the steroid is chosen from budesonide, mometasone, triamcinolone, fluticasone, and combinations thereof.

In some aspects, the techniques described herein relate to a kit, wherein: the sodium bicarbonate is contained within the first anhydrous dosing compartment; and the steroid is contained within the second anhydrous dosing compartment.

In some aspects, the techniques described herein relate to a kit further including a first sachet with one or more anhydrous dosing compounds, the first sachet containing sodium bicarbonate within the one or more anhydrous dosing compartments of the first sachet, and the kit including a second sachet containing sodium chloride and a steroid within the one or more anhydrous dosing compartments of the second sachet, the first sachet and the second sachet being separate.

In some aspects, the techniques described herein relate to a kit, wherein: the plurality of anhydrous active ingredients includes a sodium bicarbonate, an antihistamine, and a sodium chloride.

In some aspects, the techniques described herein relate to a kit for intranasal administration of a composition, the kit including: a first anhydrous dosing compartment and a second anhydrous dosing compartment separated by a middle barrier layer, the first anhydrous dosing compartment containing from about 300 milligrams to about 1000 milligrams of sodium bicarbonate; the second anhydrous dosing compartment containing from about 1400 milligrams to about 2500 milligrams of sodium chloride, from about 200 micrograms to about 500 micrograms of a steroid, and from about 250 micrograms to about 50000 micrograms of an antihistamine; and an opening path for opening both the first anhydrous dosing compartment and the second anhydrous dosing compartment simultaneously.

Numerous objects, features, and advantages of the embodiments set forth herein will be readily apparent to those skilled in the art upon reading of the following disclosure when taken in conjunction with the accompanying drawings.

### BRIEF DESCRIPTION OF THE DRAWINGS

FIG. 1 depicts an exemplary schematic view of a kit 100 for intranasal administration of a treatment mixture 102 according to an aspect of the present disclosure.

FIG. 2 depicts a method 200 of reducing nasal inflammation in a subject in need thereof according to an aspect of the present disclosure.

FIG. 3 depicts an schematic view of an aspect of a kit 106 according to an aspect of the present disclosure.

FIG. 4 depicts an exemplary sachet 106 according to an aspect of the present disclosure.

FIG. 5a-d depicts another exemplary sachet 106 according to an aspect of the present disclosure.

FIG. 6a-d depicts another exemplary sachet 106 according to an aspect of the present disclosure.

### DETAILED DESCRIPTION

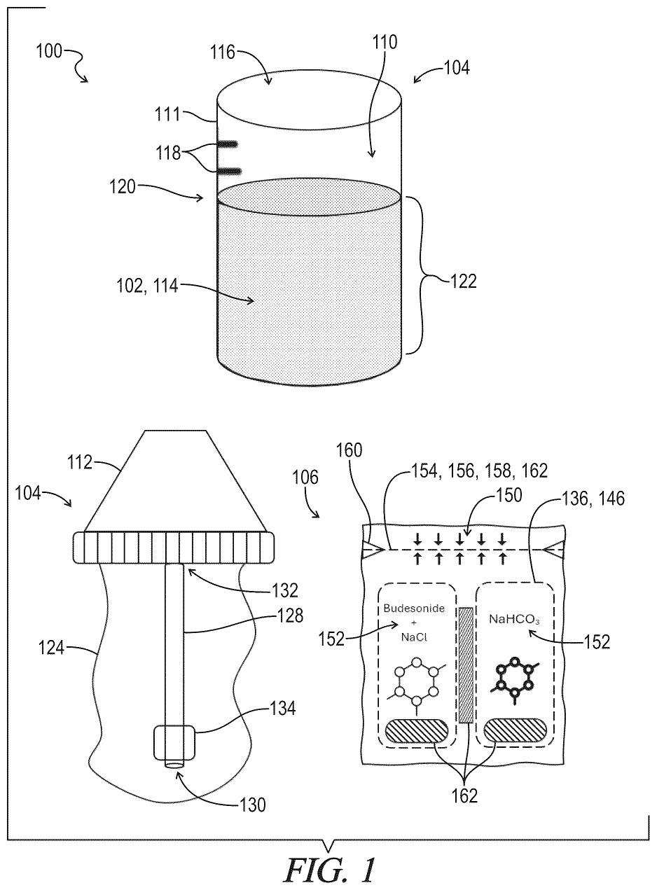

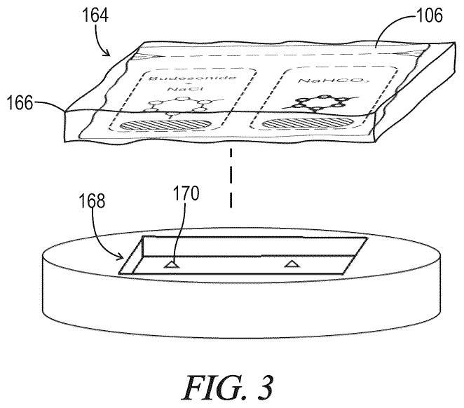

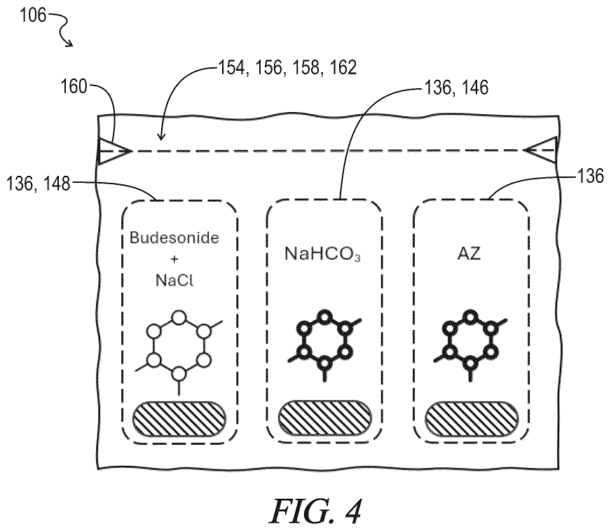

Referring now to FIGS. 1-6, various aspects may now be described of a kit for intranasal administration of a treatment mixture and a method of reducing nasal inflammation according to the present disclosure.

FIG. 1 depicts an exemplary schematic view of a kit 100 for intranasal administration of a treatment mixture 102 according to an aspect of the present disclosure. In some aspects, the kit 100 may include at least one intranasal device 104 and at least one partitioned sachet 106. In some instances, the kit 100 may include only the at least one intranasal device 104 and the at least one partitioned sachet 106. In other instances, the kit 100 may include additional components contemplated by the present disclosure.

In some aspects, the kit 100 may be used in a method 200 of reducing nasal inflammation in a subject in need thereof. The kit 100 may be designed to administer the treatment mixture 102 by combined action of at least the at least one partitioned sachet 106 and the at least one intranasal device 104.

In some instances, the at least one intranasal device 104 may be limited to a single intranasal device usable together with a plurality of the at least one partitioned sachet 106. In other instances, the kit 100 may include a plurality of intranasal devices.

Where the kit 100 includes a plurality of intranasal devices, each of the at least one intranasal device 104 may be distinctly dimensioned or constructed to suit a variety of aspects of administration of the treatment mixture 102. In an exemplary aspect, each of the at least one intranasal device 104 may be provided based on anatomical differences among desired users, including different dimensions associated with the fluid container 111, the outlet nozzle 112, or both to accommodate varying nasal sizes and shapes. In some aspects, the fluid container 111, the outlet nozzle 112, or both may be provided with a size and shape adjustment mechanism to ensure a comfortable and effective fit for diverse anatomies. In some aspects, the at least one intranasal device 104 may be provided with an ergonomic design or different materials to accommodate users with reduced hand strength or dexterity. The at least one intranasal device 104 may be provided in various sizes to provide different volumes associated with the reservoir 110.

The at least one intranasal device 104 may include a fluid container 111 which defines a reservoir 110 and may further include an outlet nozzle 112 releasably coupled with the fluid container 111. The fluid container 111 and the reservoir 110 may be configured to retain at least a volume of fluid 114 therein, with the fluid 114 being selectively retained in the reservoir 110 by the outlet nozzle 112. The outlet nozzle 112 may be releasably coupled to the fluid container 111 at an opening 116 of the fluid container 111 configured to receive the outlet nozzle 112. In an exemplary aspect, the opening 116 of the fluid container 111 may be provided with threading at an internal and/or external surface thereof, which is configured to engage with corresponding threading arranged at a corresponding mating surface of the outlet nozzle 112, thus providing a screw thread coupling. Other exemplary aspects of the releasable coupling engagement between the outlet nozzle 112 and the fluid container 111 are contemplated, including, but not limited to, snap-fit or press-fit coupling, plug seals, lug closure, snap-on spouts or flip-top lids, bayonet closures. These various coupling mechanisms may utilize structures to enhance a watertight seal, including o-rings, gaskets, sealing liners, and components formed of elastomeric materials.

In some aspects, the fluid container 111 need not be releasably coupled with the outlet nozzle 112 and may simply deliver the fluid 122 contained therein through the opening 116 of the fluid container 111. Similarly, the outlet nozzle 112 may be fixedly coupled with the fluid container 111 such that the fluid container 111, the opening 116, and the outlet nozzle 112 form a combination outlet. In an exemplary aspect, the fluid container 111 coupled with the outlet nozzle 112 may be provided as a neti pot.

The fluid container 111 may include a marker 118 arranged on an external surface thereof. In some aspects, the marker 118 may include a plurality of markers. The marker 118 may be arranged on an external surface of the fluid container 111 at a position to indicate an target fill volume 120 of the reservoir 110. The reservoir 110 may be configured to retain a volume of fluid 122. The volume of fluid 122 may be variable based on the total capacity of the reservoir 110. The marker 118 may be arranged to indicate a specific volume of fluid 122, which may be less than the total capacity of the reservoir 110. In some aspects, the position of the marker 118 may be based on a determined dosing of the treatment mixture 102. In an exemplary aspect, the marker 118 may include a first marker arranged at a first position on the fluid container 111 to indicate a first target fill volume 120 of fluid 122 for daily dosing according to an amount of ingredient in the at least one partitioned sachet 106. The fluid container 111 may further include a second marker arranged at a second position on the fluid container 111 to indicate a second target fill volume 120 of fluid 122 for more intensive treatment or flushing, where the second position corresponding to a volume of the fluid 122 greater than a volume of the fluid 122 that corresponds to the first position. It is contemplated that the marker 118 may include any number of a plurality of markers that may indicate an appropriate volume of the fluid 122 and which may be based on various aspects of treatment, including the type of fluid 122, an active ingredient of the treatment mixture 102, a treatment dosing regimen, or any other aspect of the method 200 of reducing nasal inflammation.

In some aspects, the fluid 122 may be water, distilled water, or sterile water. The fluid 122 may include isotonic saline, hypertonic saline, ringer's lactate, or a combination thereof. The fluid 122 may include additives or preservatives, including xylitol, bicarbonate, medicated additives, essential oils, or a combination thereof. In some aspects, additives and preservatives disclosed herein may be provided as one or more of the plurality of anhydrous active ingredients 138 to be added to the fluid 122. In some aspects, water in the amount of between about 200 and 300 milliliters comprises fluid 122. In some aspects, water in the amount of about 240 milliliters comprises fluid 122.

In some aspects, the fluid container 111 may include the reservoir 110 as a deformable reservoir. The deformable reservoir 110 may provide a compressible reservoir, or the reservoir 110 may be deformable under vacuum or various other external forces. Exemplary aspects of the deformable reservoir 110 may include forming the fluid container 111 of a flexible material, including a flexible plastic material, configured to elastically deform under squeezing pressure from a user. It is also contemplated that the fluid container 111 may be formed of flexible rubber material. In some aspects, the fluid container 111 may be formed of one material and the at least one intranasal device 104 may include an internal sleeve 124 of the same or a different material. Where the fluid container 111 includes the internal sleeve 124, the fluid 122 may be provided in the internal sleeve 124 and the internal sleeve 124 may be deformed to selectively expel the fluid 122. The fluid container 111, the internal sleeve 124, or both may also be formed with an accordian-style structure that includes a plurality of collapsible panels where each panel may be individually expanded or collapsed.

In some aspects, the at least one intranasal device 104 may further include a pump (not shown) configured to expel the fluid 122 from the reservoir 110 through the outlet nozzle 112. The pump (not shown) may be used as a standalone force for expelling the fluid 122 or may be used in conjunction with the deformable reservoir 110 to regulate the rate and volume of expulsion of the fluid 122.

In some aspects, the reservoir 110 may not be deformable or collapsible. In aspects where the reservoir 110 is not deformable or collapsible, the expulsion of the fluid 122 from the reservoir 110 may rely on action of the pump (not shown), or may operate according to gravity flow by manipulating an angle and position of the at least one intranasal device 104, or may utilize pressurized gas or propellant to expel the fluid 122 through the outlet nozzle 112. These methods of expulsion of the outlet nozzle 112 may be similarly applied to the at least one intranasal device 104 that includes the reservoir 110 that is deformable.

In some aspects, the outlet nozzle 112 may one or more types of nozzle configured to disperse the fluid 122 and the treatment mixture 102 in a particular manner. The outlet nozzle 112 may include a stream nozzle configured to produce a single, steady stream of liquid, a mist nozzle configured to produce a fine mist for even and gentle fluid distribution, a multi-stream nozzle with a plurality of perforations or opening configured to deliver several simultaneous streams of the fluid, a jet nozzle configured to produce a high flow rate focused stream of the fluid. The fluid 122 may further include various shapes or structures, including an angled or curved nozzle, a flared tip nozzle that includes a flared end configured to create a seal at a nasal entrance, a perforated dome nozzle, a wide-bore nozzle, or an ergonomic or custom-fit nozzle configured to be manipulated according to specific nasal shapes or sizes.

In some aspects, the at least one intranasal device 104 may further include a dip tube 128 configured to fluidly connect the outlet nozzle 112 to the reservoir 110. The dip tube 128 may be releasably coupled with the outlet nozzle 112 or a portion of the fluid container 111 in fluid communication with the outlet nozzle 112. The dip tube 128 may include an inlet 130 and an outlet 132, where the inlet is arranged to freely terminate within the reservoir 110 and the outlet 132 is arranged to releasably couple with the outlet nozzle 112. The dip tube 128 may include a length sufficient to reach all areas of the reservoir 110 and ensure all of the treatment mixture 102 is delivered. In some aspects, the dip tube 128 may include a straight tube, which may be used for administration of the treatment mixture 102 where the treatment mixture 102 maintains an upright position. The dip tube 128 may include a flexible dip tub formed of flexible materials, including silicone or polyethylene. Where the dip tube 128 includes a flexible dip tube, the dip tube 128 may further include a weight 134 coupled to the dip tube 128 and in close proximity to the inlet 130. The weight 134 is configured to maintain the inlet 130 of the dip tube 128 in a submerged position in the fluid 122. The dip tube 128 may be angled or pre-shaped to reach specific portions of the reservoir 110, which may be coupled in use with a fluid container 111 having a corresponding shape. The dip tube 128 may also include a corrugated dip tube, a vented dip tube, a telescoping dip tube, a tapered dip tube, a dip tube with multiple inlets, may incorporate various one-way valves to prevent backflow, or a combination thereof.

In some aspects, the at least one partitioned sachet 106 may include a plurality of anhydrous dosing compartments 136. Each of the plurality of anhydrous dosing compartments 136 is configured to contain an amount of an active ingredient. The at least one partitioned sachet 106 is thus configured to contain a plurality of anhydrous active ingredients 138. The at least one partitioned sachet 106 may be provided in a number of different forms, including cartridges, capsules, pods, pouches, blister packs, tubes, foil packs, strip packs, or other contemplated storage devices suitable for the plurality of anhydrous active ingredients 138.

In some aspects, the at least one partitioned sachet 106 includes an expendable sachet such that each of the at least one partitioned sachet 106 is designed for single use. The at least one partitioned sachet 106 may be formed of multi-layer laminates suitable for containing the plurality of anhydrous active ingredients 138. The at least one partitioned sachet 106 may include an outer layer including a layer configured to provide mechanical strength, heat resistance, heat sealability, printability, durability, and resistance to external environmental factors. Materials for the outer layer may include polyester (PET) and polypropylene (PP). The at least one partitioned sachet 106 may also include a middle barrier layer, including an aluminum foil or a metallized PET or biaxially oriented polypropylene (BOPP). The at least one partitioned sachet 106 may further include an inner sealant layer including low-density polyethylene (LDPE) or case polypropylene (CPP). Other materials may additionally be utilized for the partitioned sachet 106, including other polymers, coated materials, metallic foils and the like.

In some aspects, the at least one partitioned sachet 106 may include a visual or physical indicator configured to convey information associated with the plurality of anhydrous active ingredients 138 and or the method 200 to a user. In some aspects, the indicator may include an opening indicator mark 150 as a symbol, arrow, or other visual cue printed or etched on the at least one partitioned sachet 106 to show the correct area for tearing or opening of the at least one partitioned sachet 106. In some aspects, the indicator may also include a formulation indication 152 conveying the component and dosage of the plurality of anhydrous active ingredients 138 contained within the at least one partitioned sachet 106. In an exemplary aspect, the at least one partitioned sachet 106 includes a tear line indication for the opening indicator mark 150 and a plurality of indications identifying the formulation indication 152. As disclosed herein, the formulation indication 152 may indicate which individual one of the plurality of anhydrous dosing compartments 136 includes the sodium bicarbonate 140, the steroid 142, the sodium chloride 144, and the packaged dosage for each.

In some aspects, the indicator may include a structural aid 154 to ensure preservation and proper dosing of the plurality of anhydrous active ingredients 138 upon opening of the at least one partitioned sachet 106. The at least one partitioned sachet 106 including the structural aid 154 may provide a rupturable design. In an exemplary aspect, the structural aid 154 of the at least one partitioned sachet 106 may include a tear feature 156 as a notch, perforation, weakened wall space, or other subtractive surface feature configured to facilitate tearing at or along a position of the at least one partitioned sachet 106. The tear feature 156 may include a opening path 158 formed as a perforated line across a portion of the at least one partitioned sachet 106. The opening path 158 may be accompanied by a tear notch 160 arranged at one or both terminal ends of the opening path 158. In other aspects, the tear feature may be a location where a user may be directed to cut open sachet 106 and open at least first anhydrous dosing compartment 146 and second anhydrous dosing compartment 148 simultaneously. Otherwise stated, tear feature 156 could be a perforated line or a location where a user could cut open sachet 106.

In some aspects, the structural aid 154 may include at least one frangible seal 162 or other frangible barrier to provide a localized portion of the at least one partitioned sachet 106 that has a relatively thin wall thickness relative to the remainder portions of the at least one partitioned sachet 106. The at least one frangible seal 162 may be arranged between an internal and an external surface of the at least one partitioned sachet 106 to provide a rupturable barrier for release of the plurality of anhydrous active ingredients 138 from the at least one partitioned sachet 106. The at least one frangible seal 162 may further be arranged internally among each of the plurality of anhydrous dosing compartments 136 such that the at least one frangible seal 162 is present between internal surfaces of the at least one partitioned sachet 106. In an exemplary aspect, the at least one partitioned sachet 106 may include a first anhydrous dosing compartment 146 and a second anhydrous dosing compartment 148, with the at least one frangible seal 162 arranged between the first anhydrous dosing compartment 146 and the second anhydrous dosing compartment 148. In this exemplary aspect, the at least one frangible seal 162 may provide for an initial mixing of all of the plurality of anhydrous active ingredients 138 in the at least one partitioned sachet 106 by transforming the first anhydrous dosing compartment 146 and the second anhydrous dosing compartment 148 into a combined single one of the plurality of anhydrous dosing compartments 136, followed by opening of the at least one partitioned sachet 106 via the opening path 158 and/or tear notch 160 to expel the mixed plurality of anhydrous active ingredients 138.

Various other arrangements of the at least one frangible seal 162 may be contemplated to facilitate ordered and sequential mixing of the plurality of anhydrous active ingredients 138 and administration thereof. In a further exemplary aspect, the at least one frangible seal 162 may only be arranged among the plurality of anhydrous dosing compartments 136 while other structures of the structural aid 154 may be used between the interior and exterior of the at least one partitioned sachet 106. In a further exemplary aspect, each of the plurality of anhydrous dosing compartments 136 includes a corresponding one of the at least one frangible seal 162 such that each of the plurality of anhydrous dosing compartments 136 may be individually ruptured to administer the one or more of the plurality of anhydrous active ingredients 138 packaged therein.

The plurality of anhydrous active ingredients 138 may include a sodium bicarbonate 140, a steroid 142, a sodium chloride 144, or a combination thereof. The sodium chloride 144 may be provided as a sodium chloride buffer that is chemically inert and nonreactive with the other of the plurality of anhydrous active ingredients 138. In an exemplary aspect, the sodium chloride 144 may be a table salt (NaCl). The steroid 142 may include budesonide, mometasone, triamcinolone, fluticasone, or a combination thereof. The particular steroid 142 may be determined based on anti-inflammatory characteristics, mechanism of action, route of administration, efficacy, safety profile, potency, onset of action, bioavailability, or a combination thereof. The sodium bicarbonate 140 may be provided to readily react with any acidic components of the plurality of anhydrous active ingredients 138 to produce carbon dioxide (CO2) as a byproduct. In some aspects, the sodium chloride to sodium bicarbonate ratio may be about three to about one. Each of the plurality of anhydrous active ingredients 138 may be provided as a dry power formulation within the at least one partitioned sachet 106.

As disclosed herein, the steroid may be in a range of about 200 micrograms to about 5,000 micrograms. In various aspects, mometasone may be included as the steroid in a range from about 200 micrograms to 4,000 micrograms; fluticasone may be included as the steroid in a range from about 200 micrograms to 4,000 micrograms; budesonide may be included as the steroid in a range from about 250 micrograms to 5,000 micrograms; and triamcinolone may be included as the steroid in a range from about 200 micrograms to 4,000 micrograms. In various aspects, the steroids may be included individually or in combination.

In some aspects, the plurality of anhydrous dosing compartments 136 may include a first anhydrous dosing compartment 146 and a second anhydrous dosing compartment 148. In some aspects, each of the at least one partitioned sachet 106 of the kit 100 may be limited to the first anhydrous dosing compartment 146 and the second anhydrous dosing compartment 148 to the exclusion of any other anhydrous dosing compartments. In some aspects, each of the at least one partitioned sachet 106 may include a number of compartments of the plurality of anhydrous dosing compartments 136 individually corresponding to the number of the plurality of anhydrous active ingredients 138 such that each ingredient of the plurality of anhydrous active ingredients 138 is arranged in a separate compartment of the plurality of anhydrous dosing compartments 136.

In an exemplary aspect, the first anhydrous dosing compartment 146 includes the sodium bicarbonate 140 and the second anhydrous dosing compartment 148 includes the steroid 142 and the sodium chloride 144. For some formulations associated with the plurality of anhydrous active ingredients 138, it is preferred that the steroid 142 be arranged in one compartment of the plurality of anhydrous dosing compartments 136 and the sodium bicarbonate 140 be arranged in a different compartment of the plurality of anhydrous dosing compartments 136 such that the steroid 142 and the sodium bicarbonate 140 are contained within the at least one partitioned sachet 106 separate from each other. Ensuring this separated arrangement of at least the steroid 142 and the sodium bicarbonate 140 ensures long term chemical stability and compatibility of the plurality of anhydrous active ingredients 138. Where the steroid 142 and the sodium chloride 144 are arranged in a same compartment of the plurality of anhydrous dosing compartments 136, the sodium chloride 144 may function to ensure proper dosing of the steroid 142 as disclosed further herein.

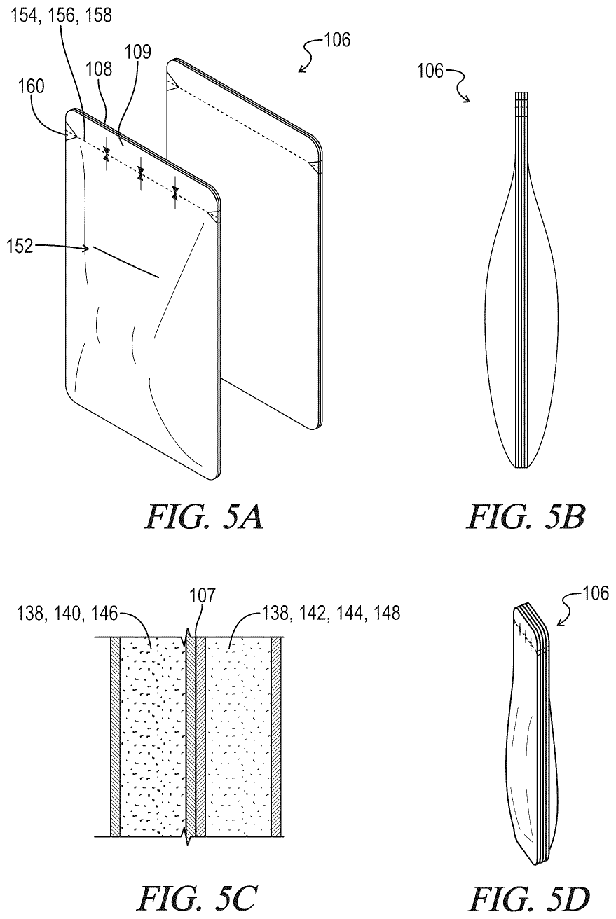

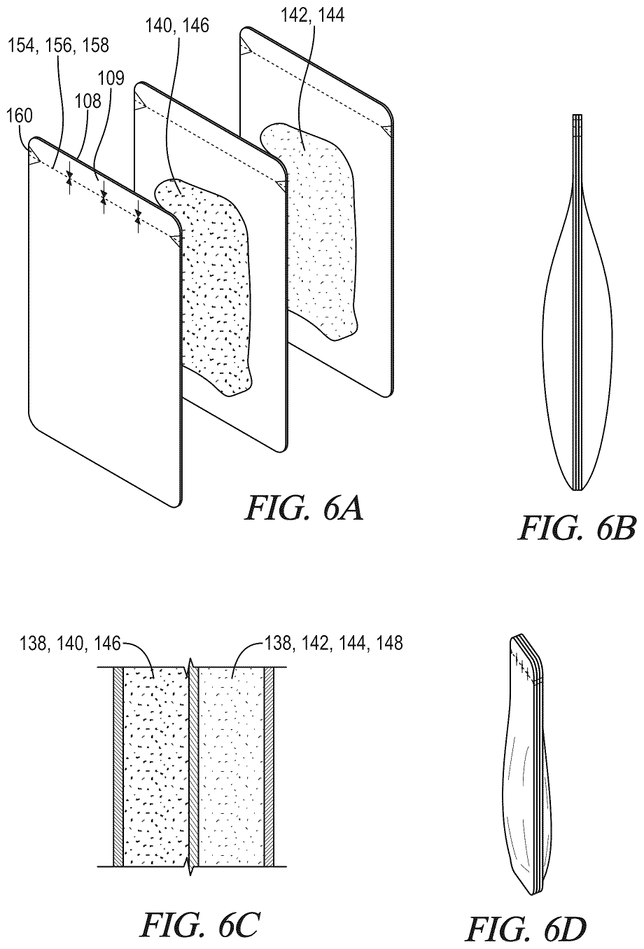

FIGS. 5a-6d depict another exemplary sachet according to an aspect of the present disclosure. In some aspects, sachet 106 may include first anhydrous dosing compartment 146 and second anhydrous dosing compartment 148 separated by a middle barrier layer 107. Middle barrier layer 107 may be of a single layer or multiple layers. For example, first anhydrous dosing compartment 146 and second anhydrous dosing compartment 148 may be formed separately with the plurality of anhydrous active ingredients 138 sealed within the different dosing compartments. The first anhydrous dosing compartment 146 and the second anhydrous dosing compartment 148 may then be attached together to form middle barrier layer 107. Various methods may be used to attach first anhydrous dosing compartment 146 to second anhydrous dosing compartment 148, including different adhesives, and the like. As such, whereas first anhydrous dosing first anhydrous dosing compartment 146 and second anhydrous dosing compartment 148 may initially be formed separately, upon adhering first anhydrous dosing compartment 146 and second anhydrous dosing compartment 148 together, sachet 106 is created with middle barrier 107. In some other aspects, first anhydrous dosing compartment 146 and second anhydrous dosing compartment 148 may be individual and not attached together. As such, there would be two separate compartments with the contents completely separated and a user could open each of the dosing compartments individually as so desired.

In such aspects, opening path 158, may be positioned along short edge 108 of sachet 106. In some aspects, opening path 158 may be beneath sealed area 109 adjacent to short edge 108 of sachet 106. When a user opens this exemplary sachet 106, via opening path 158, the user opens both first anhydrous dosing compartment 146 and second anhydrous dosing compartment 148 simultaneously. Thus, if the first anhydrous dosing compartment 146 includes the sodium bicarbonate 140 and the second anhydrous dosing compartment 148 includes the steroid 142 and the sodium chloride 144, a user opening such exemplary sachet 106 would allow for administration of sodium bicarbonate 140, steroid 142, and sodium chloride 144 simultaneously. Such design may discourage a user from opening either first anhydrous dosing compartment 146 and second anhydrous dosing compartment 148 separately as using opening path 158 would result in both compartments being opened at the same time. Furthermore, having opening path 158 below sealed area 109 adjacent to short edge 108, will further assist to maintain the structure where first anhydrous dosing compartment 146 is immediately on top of (or a mirror image to) second anhydrous dosing compartment 148 so that the user will simultaneously open both compartments. In additional aspects, further dosing components may be attached together with a single tear feature 156 being used to simultaneously open all of the dosing components togethers. As discussed herein, this may be beneficial when additional ingredients are desired to be dosed to the end user, but separated from other ingredients contained in different dosing components.

In further aspects, an example of sachet 106 with first anhydrous dosing compartment 146 and second anhydrous dosing compartment 148, may include middle barrier layer 107 as a single layer. For example, sachet 106 may be formed from a tubular-like material, with middle barrier layer 107 inserted there between to form first anhydrous dosing compartment 146 and second anhydrous dosing compartment 148. In such aspects, ingredients 138 would also be separated. Other ways of forming sachet 106 may be used, including for example the layering of sheets to form the first anhydrous dosing compartment 146 and the second anhydrous dosing compartment 148 with middle barrier layer 107 therebetween. In such aspects, sachet 106 may still function so that the tear feature 156 operates so as to open both the first anhydrous dosing compartment 146 and the second anhydrous dosing compartment 148 at about the same time. Advantageously, this results in a user having a greater degree of likelihood in administering the entirety of ingredients 138, as there is significantly less likelihood a user would erroneously open only the first anhydrous dosing compartment 146 or the second anhydrous dosing compartment 148 alone. In additional aspects, more than two dosing components could be created as well, via the use of additional middle barrier layers 107 within a tubular-like material or via the use of layers of sheets to be affixed together to form multiple dosing compartments.

In some aspects, the plurality of anhydrous active ingredients 138 may include an antihistamine 172. In an exemplary aspect, the antihistamine 172 may include azelastine or olopatadine. In various aspects, azelastine may be included in the range from about 250 micrograms to about 16,000 micrograms and olopatadine may be included in the range from about 650 micrograms to about 50,000 micrograms. The antihistamine 172 may be arranged in a compartment of the plurality of anhydrous dosing compartments 136 that is separate from the compartments containing the other of the plurality of anhydrous active ingredients 138. In an exemplary aspect, the at least one partitioned sachet 106 may include the first anhydrous dosing compartment 146 may include the sodium bicarbonate 140, the second anhydrous dosing compartment 148 may include the steroid 142 and the sodium chloride 144, and a third anhydrous dosing compartment may include the antihistamine 172. In other aspects, the antihistamine 172 or other ones of the plurality of anhydrous active ingredients 138 may be arranged in a compartment of the plurality of anhydrous dosing compartments 136 that is the same compartment as other ones of the plurality of anhydrous dosing compartments 136. In some various aspects, the antihistamine may be included without a steroid or may be included with the steroid.

In some aspects, the sodium chloride may be included in the range from about 1400 milligrams to about 2500 milligrams and may be combined with: mometasone in the range from about 200 micrograms to 4,000 micrograms; fluticasone in the range from about 200 micrograms to 4,000 micrograms; budesonide in the range from about 250 micrograms to 5,000 micrograms; and/or triamcinolone in the range from about 200 micrograms to 4,000 micrograms.

In some aspects, the sodium chloride may be included in the range from about 1400 milligrams to about 2500 milligrams and may be combined with: azelastine in the range from about 250 micrograms to 16,000 micrograms; and/or olopatadine in the range from about 650 micrograms to 50,000 micrograms.

In some aspects, the sodium chloride may be included in the range from about 1400 milligrams to about 2500 milligrams and may be combined with: mometasone in the range from about 200 micrograms to 4,000 micrograms plus olopatadine in the range from about 650 micrograms to 50,000 micrograms; mometasone in the range from about 200 micrograms to 4,000 micrograms plus azelastine in the range from about 250 micrograms to 16,000 micrograms; budesonide in the range from about 250 micrograms to 5,000 micrograms plus olopatadine in the range from about 650 micrograms to 50,000 micrograms; budesonide in the range from about 250 micrograms to 5,000 micrograms plus azelastine in the range from about 250 micrograms to 16,000 micrograms; fluticasone in the range from about 200 micrograms to 4,000 micrograms plus azelastine in the range from about 250 micrograms to 16,000 micrograms; fluticasone in the range from about 200 micrograms to 4,000 micrograms plus olopatadine in the range from about 650 micrograms to 50,000 micrograms; triamcinolone in the range from about 200 micrograms to 4,000 micrograms plus azelastine in the range from about 250 micrograms to 16,000 micrograms; and/or triamcinolone in the range from about 200 micrograms to 4,000 micrograms plus olopatadine in the range from about 650 micrograms to 50,000 micrograms.

In some aspects, the plurality of anhydrous active ingredients 138 may include various dosages by weight of an effective amount of each of the ingredients, where the effective amount of the sodium bicarbonate 140 being a sodium bicarbonate dosage, the effective amount of the steroid 142 being a steroid dosage, and the effective amount of the sodium chloride 144 being a sodium chloride dosage. The steroid 142 and the sodium chloride 144 together may be provided in a combined effective amount together. Where the steroid 142 and the sodium chloride 144 are arranged in the same compartment of the plurality of anhydrous dosing compartments 136, the steroid 142 may be provided in a relatively small effective amount, with the remainder of the dosage of that compartment being provided by the sodium chloride 144. The sodium bicarbonate 140 may be provided in an effective amount of about 300 milligrams to 1000 milligrams, and in some aspects about 700 milligrams.

In further exemplary aspects, the effective amount of each of the plurality of anhydrous active ingredients 138 may vary according to desired treatment effect, level of inflammation in a subject in need of treatment, and other characteristics associated with a subject. In some aspects, the steroid 142 may be provided in an effective amount as low as about 1 microgram, though may range from about 200 micrograms to 4000 micrograms, allowing for minor fluctuations in potency or blending uniformity during production. Additional ranges, such as 0.75 milligrams to 1.25 milligrams or 0.9 milligrams to 1.1 milligrams, may also be utilized, depending on the desired therapeutic effect or formulation requirements. The sodium chloride 144 may be included at approximately 1400 milligrams to about 2500 milligrams. In some aspects, sodium chloride 144 may be included at about 2300 milligrams. In additional aspects, sodium chloride 144 may be adjusted between about 2250 milligrams to 2350 milligrams to account for isotonicity adjustments for physiological compatibility. Narrower ranges, such as 2280 milligrams to 2320 milligrams, may be applied to optimize tonicity for specific use cases. Broader ranges, such as 2200 milligrams to 2400 milligrams, may also be appropriate for multi-purpose formulations or to accommodate excipient-grade variability. The sodium bicarbonate 140 may be included at approximately 300 milligrams to 1000 milligrams. In some aspects sodium bicarbonate 140 may be at about 700 milligrams, or may range from about 650 milligrams to 750 milligrams to ensure effective buffering capacity and pH control. Narrower ranges, such as 680 milligrams to 720 milligrams, may be preferred for cases where pH precision is critical. In some aspects, ranges such as 600 milligrams to 800 milligrams may be employed for more generalized formulations, particularly where environmental storage conditions could influence stability. In some aspects, the steroid dosage will be between about 200 micrograms and 5000 micrograms depending upon the selected steroid. The various effective amounts disclosed for each of the plurality of anhydrous active ingredients 138 may be combined in various permutations to allow for flexibility in dosing of an effective amount, desired therapeutic effect, and general desired balance between steroid administration and nasal irrigation.

In some aspects, the at least one partitioned sachet 106 may include a cartridge 164. The cartridge 164 may include various structural distinctions compared to the at least one partitioned sachet 106 formed as a tearable pouch, with these distinctions facilitating insertion and retention of the cartridge 164 in the at least one intranasal device 104 and automatic release of the plurality of anhydrous active ingredients 138 based on physical interaction of the cartridge 164 with one or more components of the at least one intranasal device 104. The cartridge 164 may be specifically designed for insertion and retention in the at least one intranasal device 104. In some aspects, the cartridge 164 may include a relatively thicker material or reinforcements of the at least one partitioned sachet 106, including an external frame 166.

The cartridge 164 may be characterized by a specified form or shape corresponding to an associated seat 168 provided as part of the at least one intranasal device 104. In an exemplary aspect, the fluid container 111, the outlet nozzle 112, or a combination thereof may include the associated seat 168. The associated seat 168 may include one or more of a retaining wall, a depression dimensioned to correspond to the at least one partitioned sachet 106, or other features configured to fix a position of the at least one partitioned sachet 106 when inserted into the associated seat 168. The associated seat 168 may include puncture feature 170 configured to puncture the at least one partitioned sachet 106 where the at least one partitioned sachet 106 is releasably retained in the associated seat 168. In an exemplary aspect, the puncture feature 170 may include a pointed or bladed protrusion projecting from the associated seat 168. The cartridge 164 may be inserted into the associated seat 168 and aligned within the seat according to various alignment structures associated with the seat, where upon coupling of the outlet nozzle 112 with the fluid container 111 the puncture feature 170 may puncture the at least one partitioned sachet 106 and release the plurality of anhydrous active ingredients 138 into the reservoir 110. In further aspects, the at least one intranasal device 104 may include a puncture button (not shown) arranged to facilitate puncture of the at least one partitioned sachet 106.

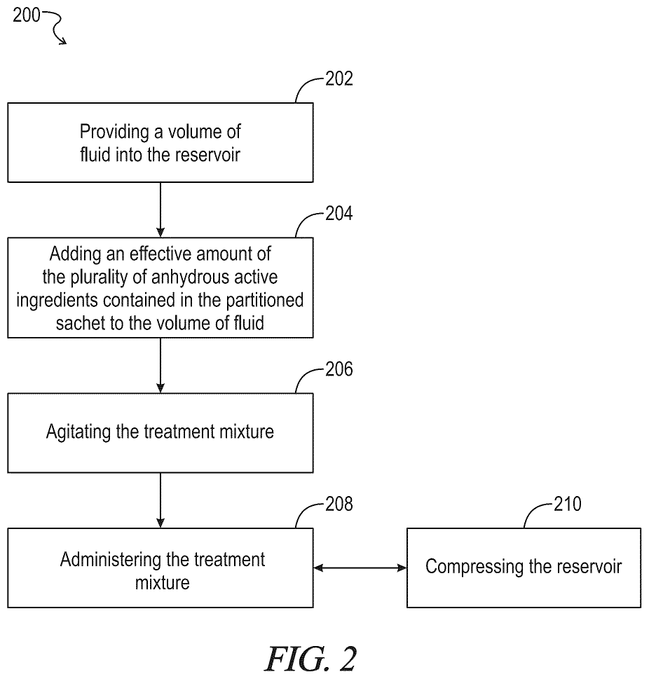

FIG. 2 depicts the method 200 of reducing nasal inflammation in a subject in need thereof according to an aspect of the present disclosure. In some aspects, the subject has been diagnosed as having a nasal inflammatory condition prior to performing the method 200. In some exemplary aspects, the nasal inflammatory condition includes acute sinusitis, chronic rhino sinusitis, allergic rhinitis, chronic rhinitis, or combinations thereof.

In some aspects, the method 200 may include a step of providing 202 a volume of the fluid 122 into the reservoir 110. The providing 202 step may include providing the fluid 122 in a volume corresponding to the marker 118 arranged at the fluid container 111, the reservoir 110, or both. The providing 202 may include removing the outlet nozzle 112 from the fluid container 111 to provide an inlet into the reservoir 110. In an exemplary aspect, the step of providing 202 the fluid 122 may include providing about 240 ml of the fluid 122.

The method 200 may continue with a step of adding 204 an effective amount of the plurality of anhydrous active ingredients 138 contained in the at least one partitioned sachet 106 to the volume of fluid 122 to form a treatment mixture. The adding 204 may be performed in a simultaneous or sequential manner based on the at least one partitioned sachet 106, the plurality of anhydrous active ingredients 138, or any other aspect of the kit 100 that may influence the administration of the treatment mixture 102. In an exemplary aspect, the adding 204 the effective amount of the plurality of anhydrous active ingredients 138 includes simultaneously adding all of the plurality of anhydrous active ingredients 138 to the fluid 122 to form the treatment mixture. Where the step of adding 204 the plurality of anhydrous active ingredients 138 to the fluid 122 is performed in a simultaneous manner, the puncture of the cartridge 164 via the puncture feature 170 may facilitate simultaneous addition of the plurality of anhydrous active ingredients 138 to the fluid 122.

Where the adding 204 step is performed in a sequential manner, the sequential addition of the plurality of anhydrous active ingredients 138 may be performed by first adding one or more of the plurality of anhydrous active ingredients 138 from the first anhydrous dosing compartment 146 and subsequently adding others of the plurality of anhydrous active ingredients 138 from the second anhydrous dosing compartment 148 or any other compartment of the plurality of anhydrous dosing compartments 136.

In some aspects, the step of adding 204 the effective amount of the plurality of anhydrous active ingredients 138 includes simultaneously adding at least the sodium bicarbonate 140, the steroid 142, and the sodium chloride 144. In some aspects, the step of adding 204 the effective amount of the plurality of anhydrous active ingredients 138 includes first adding the sodium bicarbonate 140 to the fluid 122 and subsequently adding the steroid 142 and the sodium chloride 144 to the fluid 122, the combination resulting in a treatment mixture.

In some aspects, the method 200 may also include a step of agitating 206 the treatment mixture 102 to form a solution, a solute, or a suspension. The agitating 206 step may include shaking, stirring, increasing temperature of the fluid 122, adjusting pH of the treatment mixture, adding on or more co-solvents or solubilizing agents, applying ultrasonic waves to the treatment mixture, or a combination thereof. The treatment mixture 102 includes the plurality of anhydrous active ingredients 138 as added to the fluid 122 to form at least the treatment mixture. In some aspects, the treatment mixture 102 will be delivered in the treatment mixture in the form of a solution, suspension, or solute.

After the plurality of anhydrous active ingredients 138 have been brought into a solution, suspension, or solute to form the treatment mixture 102, the method 200 may continue with a step of administering 208 the treatment mixture 102 from the fluid container 111 through the outlet nozzle 112 to at least the nasal cavity of a subject. In some aspects, the treatment mixture 102 may be administered to other target areas of application of the subject, including the paranasal sinuses. In some aspects, the step of administering 208 the treatment mixture 102 may include compressing 210 the reservoir 110 to administer the treatment mixture 102 through the outlet nozzle 112 in a nasal vestibule of the subject. In other aspects, the step of administering 208 the treatment mixture 102 may include tilting the at least one intranasal device 104 or applying pressure from the pump (not shown) to administer the treatment mixture 102 through the outlet nozzle 112.

In some aspects where the at least one partitioned sachet 106 is provided as a cartridge 164, the method 200 may include positioning the cartridge 164 in the associated seat 168 at the at least one intranasal device 104 before the step of adding 204 the plurality of anhydrous active ingredients 138 to the fluid 122.

In optional aspects, the plurality of anhydrous active ingredients 138 may be provided to the at least one partitioned sachet 106, where the at least one partitioned sachet 106 includes at least the first anhydrous dosing compartment 146 and the second anhydrous dosing compartment 148. In some aspects, the plurality of anhydrous dosing compartments 136 may comprise at least one dosing compartment per active ingredient of the plurality of anhydrous active ingredients 138. Such may also include a step of providing the fluid container 111, which is configured to deliver the treatment mixture.

Other steps in the aspect may include providing a dosage for each of the plurality of anhydrous active ingredients 138, including a sodium bicarbonate 140 dosage, a steroid 142 dosage, and a sodium chloride 144 dosage. Additionally, optional aspects may include providing an antihistamine 172 dosage if administration of the antihistamine 172 is desired.

In some aspects, a method may include providing the sodium bicarbonate 140 dosage to the first anhydrous dosing compartment 146 of the at least one partitioned sachet 106 and providing the sodium chloride 144 dosage to the second anhydrous dosing compartment 148 of the at least one partitioned sachet 106. Other aspects may further include providing the steroid 142 dosage to the second anhydrous dosing compartment 148 along with the sodium chloride 144 dosage, but may exclude providing the steroid 142 dosage to the first anhydrous dosing compartment 146 with the sodium bicarbonate 140 dosage.

In some other aspects, the method may include providing a plurality of the at least one intranasal device 104 in a set or recommended regimen and further including the fluid container 111 along with the plurality of the at least one intranasal device 104, thus providing the kit 100. In some aspects, this method may provide the kit 100 without providing the outlet nozzle 112 of the fluid container 111 in instances where simplicity of the fluid container 111 is desired as a unitary vessel.

As used herein, the phrase “one or more of,” when used with a list of items, means that different combinations of one or more of the items may be used and only one of each item in the list may be needed. For example, “one or more of” item A, item B, and item C may include, for example, without limitation, item A or item A and item B. This example also may include item A, item B, and item C, or item B and item C.

As used herein, the phrase “at least” and “at least one” shall mean one or more unless explicitly stated otherwise. For example, “at least one component” refers to one component, two components, or more than two components. The term “at least one” encompasses all singular and plural variations of the referenced element unless the context dictates otherwise. The phrase “at least one of A, B, or C” shall mean one or more of A, one or more of B, one or more of C, or any combination thereof. Unless explicitly stated otherwise, this phrase does not require the inclusion of all listed elements.

The terms “about” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values. Numerical quantities given herein are approximate unless stated otherwise, meaning that the term “about” or “approximately” can be inferred when not expressly stated.

Thus, it is seen that the apparatus and methods of the present disclosure readily achieve the ends and advantages mentioned as well as those inherent therein. While certain aspects of the disclosure have been illustrated and described for present purposes, numerous changes in the arrangement and construction of parts and steps may be made by those skilled in the art, which changes are encompassed within the scope and spirit of the present disclosure as defined by the appended claims. Each disclosed feature or aspect may be combined with any of the other disclosed features or aspects.

1. A method for providing a nasal treatment, comprising:
providing a partitioned sachet containing a plurality of anhydrous active ingredients, wherein the partitioned sachet comprises:
a first anhydrous dosing compartment and a second anhydrous dosing compartment separated by a middle barrier layer, and the plurality of active ingredients are contained within the first anhydrous dosing compartment and the second anhydrous dosing compartment, and
a tear feature;
engaging the tear feature to simultaneously open the first anhydrous dosing compartment and the second anhydrous dosing compartment;
adding the plurality of anhydrous active ingredients to a volume of fluid to form a treatment mixture; and
administering the treatment mixture to an individual, wherein the treatment mixture is in the form of a solution or suspension.

2. The method of claim 1, wherein:
the first anhydrous dosing compartment comprises a sodium bicarbonate dosage and the second anhydrous dosing compartment comprises a steroid dosage and sodium chloride dosage.

3. (canceled)

4. (canceled)

5. The method of claim 2, wherein adding the plurality of anhydrous active ingredients to the volume of fluid comprises simultaneously pouring out the sodium bicarbonate dosage of the first anhydrous dosing compartment and the steroid dosage, and sodium chloride dosage of the second anhydrous dosing compartment through the opening in the first anhydrous dosing compartment and the second anhydrous dosing compartment created by engaging the tear feature.

6. The method of claim 2, wherein:
the sodium bicarbonate dosage is between about 300 milligrams and 1000 milligrams; and
the sodium chloride dosage is between about 1400 milligrams and 2500 milligrams.

7. The method of claim 6, wherein the sodium chloride dosage and the sodium bicarbonate dosage are present in a weight ratio of about 3:1.

8. The method of claim 2, wherein the steroid dosage is selected from the group consisting of budesonide, mometasone, triamcinolone, fluticasone, and combinations thereof.

9. The method of claim 8, wherein the steroid dosage is between about 200 micrograms and about 5000 micrograms.

10. The method of claim 2, further comprising an antihistamine in a range between about 250 micrograms to about 50000 micrograms.

11. The method of claim 10, wherein the antihistamine is selected from the group consisting of azelastine, olopatadine, and combinations thereof.

12. (canceled)

13. A kit for intranasal administration of a composition, the kit comprising:
at least one partitioned sachet comprising:
a first anhydrous dosing compartment and a second anhydrous dosing compartment separated by a middle barrier layer, and the plurality of active ingredients are contained within the first anhydrous dosing compartment and the second anhydrous dosing compartment, and
a tear feature that, when engaged, simultaneously opens the first anhydrous dosing compartment and the second anhydrous dosing compartment;
wherein the composition comprises a plurality of anhydrous active ingredients.

14. (canceled)

15. The kit of claim 13, wherein:
the plurality of anhydrous active ingredients comprises sodium bicarbonate, a steroid, and sodium chloride.

16. The kit of claim 15, wherein:
the steroid is selected from the group consisting of budesonide, mometasone, triamcinolone, fluticasone, and combinations thereof.

17. The kit of claim 15, wherein:
the sodium bicarbonate is contained within the first anhydrous dosing compartment; and
the steroid is contained within the second anhydrous dosing compartment.

18. (canceled)

19. The kit of claim 13, wherein:
the plurality of anhydrous active ingredients comprises a sodium bicarbonate, an antihistamine, and sodium chloride.

20. A partitioned sachet for intranasal administration of a composition, the partitioned sachet comprising:
a first anhydrous dosing compartment and a second anhydrous dosing compartment separated by a middle barrier layer,
the first anhydrous dosing compartment containing from about 300 milligrams to about 1000 milligrams of sodium bicarbonate;
the second anhydrous dosing compartment containing from about 1400 milligrams to about 2500 milligrams of sodium chloride, from about 200 micrograms to about 5000 micrograms of a steroid, and from about 250 micrograms to about 50000 micrograms of an antihistamine; and
a tear feature for opening both the first anhydrous dosing compartment and the second anhydrous dosing compartment simultaneously.

21. The kit of claim 17, further comprising sodium chloride contained in the second anhydrous dosing compartment.

22. The kit of claim 17, wherein the first anhydrous dosing compartment comprises from about 300 milligrams to about 1000 milligrams of sodium bicarbonate.

23. The kit of claim 17, wherein the second anhydrous dosing compartment comprises from about 200 micrograms to about 5000 micrograms of the steroid.

24. The kit of claim 21, wherein the second anhydrous dosing compartment comprises from about 1400 milligrams to about 2500 milligrams of sodium chloride.
